(19) United States
(12) Patent Application Publication
(10) Pub. No.: US 2008/0097486 A1
(43) Pub. Date: Apr. 24, 2008

(54) Systems and Methods for Direct Restoration of Foraminal Volume

(76) Inventor: Anthony C. Ross, Charleston, SC (US)

Correspondence Address: JONATHAN SPANGLER;NU VASIVE, INC., 4545 TOWNE CENTRE COURT, SAN DIEGO, CA, 92121, US

(21) Appl. No.: 11/664,707

(22) PCT Filed: Oct. 6, 2005
(86) PCT No.: PCT/US05/36384

§ 371 Date: Apr. 3, 2007

Related U.S. Application Data
(60) Provisional application No. 60/616,758, filed on Oct. 6, 2004.

(51) Int. Cl. A61B 17/08; A61B 17/58

(52) U.S. Cl. 606/151; 606/61

(57) ABSTRACT

Systems and methods for directly restoring the volume of one or more intervertebral foramen (IVF) within a spine.

### BACKGROUND OF THE INVENTION

I. Field of the Invention

The present invention relates generally to spinal surgery and, more particularly, to systems and methods for directly restoring the volume of one or more intervertebral foramen (IVF) within a spine.

II. Discussion of the Prior Art

An intervertebral foramen (IVF) is an aperture extending generally laterally from the spinal canal dimensioned to accommodate the exiting nerve roots from the spinal chord at a given vertebral level in the spine. Within a single vertebral level, an IVF is defined anteriorly by the annulus of the intervertebral disc and the posterior and lateral aspects of the adjacent superior and inferior vertebral bodies, superiorly by the pedicle of the superior vertebra, inferiorly by the pedicle of the inferior vertebra, and posteriorly by the facet joint formed by the inferior articulating facet of the superior vertebra and the superior articulating facet of the inferior vertebra. In a normal, non-pathologic spine, the IVF is dimensioned such that the exiting nerve roots are adequately protected from compression and/or other undesirable contact during flexion, extension, lateral bending, and axial rotation of the particular vertebral level. The key to this protection is to ensure that the volume of the IVF is maintained during (or promptly restored after) such movements, as well as under the axial loading which occurs at that vertebral level during activities of daily living.

The spatial integrity or volume of the IVF may become compromised due to any of a number of events or pathologies, resulting in a constriction or narrowing of one or more aspects of the IVF. These include, but are not necessarily limited to, degenerative disc disease, disc failure or rupture due to trauma, osteophyte formation and/or calcification of the ligamentum flavum, intervertebral osteochondrosis, scoliosis, and/or destabilization from spine surgery procedures (e.g. discectomy, fusion, total disc replacement, nucleus replacement, etc. . . . ). When this occurs, the resulting narrowing or constriction (“lateral stenosis”) may cause to the exiting nerve root to be compressed by one or more of the aspects which define the IVF. Depending on the severity, this may cause any of a host of problems for the patient, including shooting pain (radiculopathy) and/or impaired motor or sensory nerve function due to the nerve compression. It may also cause the superior and inferior articulating facets to become misaligned (“facet imbrication”) and/or undesirably compressed against one another, either of which may be painful and/or adversely affect the proper function of the facet joint. This, in turn, may exacerbate or further any improper equilibration of the intervertebral disc and therefore hasten the degenerative cascade.

A variety of techniques have been developed over time for treating spinal pathologies, some of which have the effect of restoring the volume of the IVF. However, all such techniques do so indirectly through the use of procedures or implants in aspects of the spine remote to or outside of the IVF. These procedures include, but are not necessarily limited to, the use of pedicle screw systems (fixed or dynamic) extending between adjacent pedicles, spinous process spacer systems positioned between adjacent spinous processes, plating systems (lateral and/or anterior) coupled between adjacent vertebral bodies, and intervertebral implants (fusion, total disc replacement, nucleus replacement) positioned between adjacent vertebral bodies. None of these systems or procedures restores the IVF volume in a direct manner. Moreover, many of these procedures involve fusing or rigidly affixing the adjacent levels of the spine, such is via interbody fusion, posterior fusion, fusion via plating and/or fusion of the facet joint itself, each of which limits normal physiologic motion.

The present invention is directed at overcoming, or at least improving upon, the disadvantages of the prior art.

### SUMMARY OF THE INVENTION

The present invention accomplishes this goal by providing systems and methods involving the introduction of at least one volume restoration element into the IVF of a patient suffering from lateral stenosis. The volume restoration element may be provided in any number of suitable shapes and sizes, and is constructed with sufficient structural characteristics to restore and maintain the volume of the IVF after implantation. Restoring the spatial integrity of the IVF according to the present invention advantageously relieves the impingement (compression) of the spinal nerves, which results in pain relief for the patient and/or improved motor and/or sensory nerve functions (if previously impaired due to nerve root compression). It also advantageously restores the facet joint to its normal or near-normal physiologic state, by unloading the facet joint and/or returning the superior and inferior articulating facets into proper position and thereby reversing or minimizing any facet imbrication. A still further advantage of the present invention is that the volume restoration element causes distraction osteogenesis over time, which means the boney aspects of the IVF which are in contact with the volume restoration element may gradually migrate or move as a result of the force being exerted by the volume restoration element. By gradually yielding to the force of the volume restoration element, the volume of the IVF may actually increase over time, further ensuring protection from nerve compression, facet imbrication, and undesirable facet loading.

The volume restoration element of the present invention may be implanted via a traditional “open” technique and/or via minimally disruptive techniques involving tissue sparing and low profile instrumentation. It may introduced into the surgical target site from all conceivable trajectories, including but not limited to a lateral approach, a posterior approach, a postero-lateral approach, an anterior approach (trans-disc space), and/or an axial (trans-sacral) approach.

The volume restoration element of the present invention is also extremely versatile, in that it may be used as a stand-alone device and in conjunction with other spinal implants. As a stand-alone device, the volume restoration element of the present invention will be the only type of device solution implanted in the patient, and may be employed in only one or IVF or the part for each vertebral level (so-called “unilateral volume restoration”) and/or in each IVF for a particular vertebral level (so-called “bi-lateral volume restoration”). The volume restoration element may be employed at a single vertebral level or in multiple vertebral levels (adjacent or non-adjacent).

When used in conjunction with other spinal implants, the volume restoration element of the present invention may advantageously supplement or augment the function of the other devices. Such other devices include, but are not necessarily limited to, total disc replacements, nucleus replacements, pedicle-based dynamic stabilization devices, spinous process based dynamic stabilization devices, and vertebral body repair devices or systems such as kyphoplasty. The volume restoration element of the present invention may also be used at vertebral levels above or below such other spinal implants, including fusion implants, to reduce the incidence of so-called adjacent level disease and/or add to the overall structural stability of the spine.

In still further aspects, the volume restoration element of the present invention may be formed as part of such other spinal implants, including but not limited to a total disc replacement, a nucleus replacement, a facet replacement system, a spinous process spacer and/or a pedicle screw system. In this manner, the particular other spinal implant may benefit from the advantages of the volume restoration element of the present invention in addition to its own.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIG. 1 is a lateral view of a volume restoration element implanted in use within an intervertebral foramen (IVF) according to the present invention;

FIG. 2 is a lateral view of a volume restoration element implanted in use within an IVF according to the present invention;

FIG. 3 is a front side view of a volume restoration element according to the present invention of the type shown in FIGS. 1-2;

FIG. 4 is a front perspective view of a volume restoration element according to the present invention of the type shown in FIGS. 1-2;

FIG. 5 is a rear perspective view of a volume restoration element according to the present invention of the type shown in FIGS. 1-2;

FIG. 6 is a back side view of a volume restoration element according to the present invention of the type shown in FIGS. 1-2;

FIG. 7 is a lateral view of a volume restoration element implanted in use within an IVF according to the present invention;

FIG. 8 is a front perspective view of a volume restoration element according to the present invention of the type shown in FIG. 7;

FIG. 9 is a lateral view of a volume restoration element implanted in use within an IVF according to the present invention;

FIG. 10 is a side view of a volume restoration element according to the present invention of the type shown in FIG. 9;

FIG. 11 is an end view of a volume restoration element according to the present invention of the type shown in FIG. 9;

FIG. 12 is a perspective view of a volume restoration element according to the present invention of the type shown in FIG. 9;

FIG. 13 is a lateral view of a volume restoration element implanted in use within an IVF according to the present invention;

FIG. 14 is a perspective view of a volume restoration element according to the present invention of the type shown in FIG. 13, in an unexpanded state;

FIG. 15 is a perspective view of a volume restoration element according to the present invention of the type shown in FIG. 13, in an expanded state;

FIG. 16 is a front perspective view of a volume restoration element according to the present invention;

FIG. 17 is a side view of a volume restoration element according to the present invention;

FIG. 18 is a back perspective view of a volume restoration element according to the present invention;

FIG. 19 is a back perspective view of a volume restoration element according to the present invention;

FIG. 20 is a back perspective view of a volume restoration element according to the present invention;

FIG. 21 is a side view of a volume restoration element according to the present invention of the type shown in FIG. 20;

FIGS. 22-23 are perspective views a volume restoration element coupled to a two-prong insertion device according to one embodiment of the present invention;

FIG. 24 is a top view of a volume restoration element coupled to a two-pronged insertion device according to an alternative embodiment of the present invention;

FIG. 25 is a perspective view of a three-pronged inserter used to insert a volume restoration element according to a still further alternative embodiment of the present invention;

FIG. 26 is a front view of a volume restoration element coupled to the insertion device of FIG. 25 in a closed position prior to insertion;

FIG. 27 is a front view of a volume restoration element coupled to the insertion device of FIG. 25 in an open position during insertion;

FIG. 28 is a recess saw according to one embodiment of the present invention, used to create a pair of recesses in adjacent vertebrae for use with the volume restoration element of the type shown in FIGS. 9-12;

FIG. 29 is a perspective view of an insertion device according to a still further embodiment of the present invention, shown without a volume restoration element of the present invention;

FIG. 30 is a perspective view of an insertion device according to the embodiment of the type shown in FIG. 29, shown with a volume restoration element loaded therein according to the present invention;

FIG. 31 is a perspective view of an insertion device according to the embodiment of the type shown in FIG. 29, shown with a spinal nerve being loaded into the device according to the present invention;

FIG. 32 is a perspective view of an insertion device according to the embodiment of the type shown in FIG. 29, shown with a spinal nerve fully loaded into the device according to the present invention;

FIG. 33 is a perspective view of an insertion device according to the embodiment of the type shown in FIG. 29, shown with volume restoration element deployed and the spinal nerve still loaded into the device according to the present invention;

FIG. 34 is a perspective view of an insertion device according to the embodiment of the type shown in FIG. 29, shown with the spinal nerve unloaded from the device according to the present invention;

FIG. 35 is a side view of a volume restoration element employed in conjunction with a nucleus replacement implant according to the present invention;

FIG. 36 is a side view of a volume restoration element employed in conjunction with a total disc replacement implant according to the present invention; and

FIG. 37 is a side view of a volume restoration element employed incorporated into the manufacture of a total disc replacement according to the present invention.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, nerous implementation-specific decisions must be made to achieve the developers' specific goals, such as compliance with system-related and business-related constraints, each will vary from one implementation to another. Moreover, it will be appreciated that a development effort might be complex and time-consuming, but would nevertheless routine undertaking for those of ordinary skill in the art having the benefit of this closure. The volume restoration element and related methods disclosed herein boasts a ety of inventive features and components that warrant patent protection, both individually and in combination.

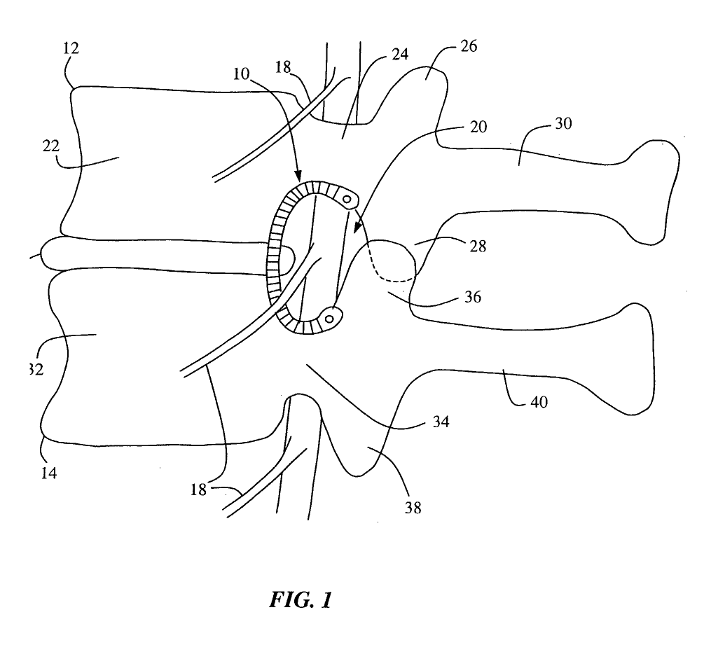

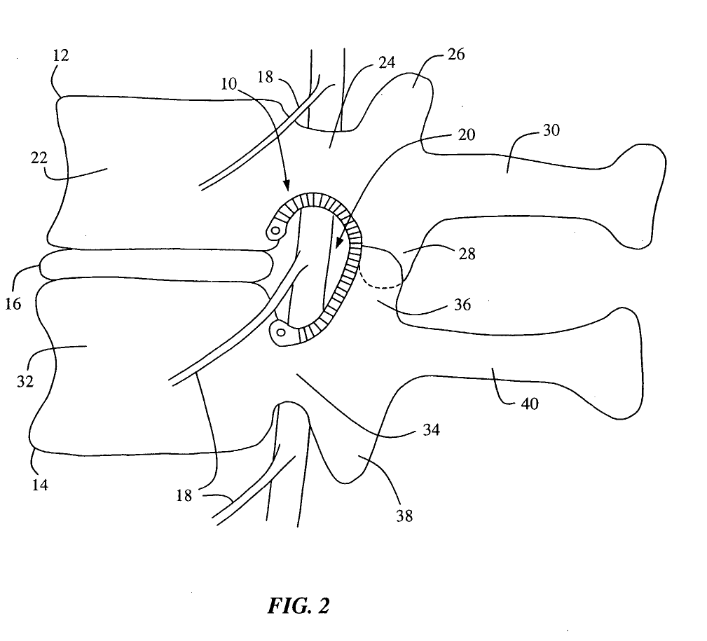

FIGS. 1-2 illustrate a volume restoration element 10 of the present invention implanted within an intervertebral foramen (IVF) 20 of a human spine. For purposes of simplicity, the volume restoration element 10 of the present invention will be described herein within the context of a single vertebral level with unilateral implantation. It will be appreciated, however, that the volume restoration element 10 of the present invention may ised in multiple vertebral levels and with bi-lateral implantation without departing from scope of the invention. The single vertebral level shown comprises a superior vertebra an inferior vertebra 14, an intervertebral disc 16, and spinal nerves 18 exiting the IVF Superior vertebra 12 includes a vertebral body 22, pedicle 24, superior facet 26, superior facet 28, and spinous process 30. Inferior vertebra 14 includes a vertebral body 32, pedicle 34, superior facet 36, inferior facet 38, and spinous process 40. The IVF 20 is ned by (described clockwise) the posterior aspect of the disc 16 (annulus), portions of posterior aspect of the superior vertebral body 22, portions the inferior aspect of the superior pedicle 24, portions of the anterior aspect of the inferior facet 28 of the superior vertebra 12, portions of the anterior aspect of the superior facet 36 of the inferior vertebra and portions of the superior aspect of the inferior pedicle 34.

According to the present invention, the volume restoration element 10 may be rted or otherwise introduced into the IVF 20 such that the volume restoration element contacts at least a portion of the periphery of the IVF 20. FIG. 1 illustrates a volume oration element 10 inserted in a generally posterior orientation such that the open end of the volume restoration element 10 is facing in a generally posterior direction with respect to the spine. FIG. 2 illustrates the volume restoration element 10 inserted in a generally anterior orientation such that the open end of the volume restoration element 10 s facing in a generally anterior direction with respect to the spine. In either orientation, the volume restoration element 10 biases the IVF 20 from a previously stenotic condition (not shown) to the condition of restored volume shown. The volume restoration element 10 accomplishes this by being constructed from any number of biologically compatible and structurally capable materials dimensioned to maintain the IVF 20 in this condition over time. Such materials may include, but are not necessarily limited to, metals (e.g. titanium, cobalt chrome, stainless steel, nitinol, etc. . . . ), plastics (e.g. PEEK, PEKK, thermopolymers) and any suitable biologically-derived compositions capable of serving as the volume restoration element 10.

Volume restoration element 10 preferably exhibits an elastic property that causes ne volume restoration element 10 to want to return to its original state once released from ne insertion device. When compressed, the volume restoration element 10 will exert an expansion force against the periphery of the UVF 20, which has the effect of either restoring ne spatial integrity or volume of the IVF 20 back to the neutral or natural physiologic state, r at least biasing the IVF 20 as far as can towards this neutral or natural physiologic state. s will be described below, this outward expansion force of the volume restoration element 10, when equipped with anti-migration features, will also serve to further engage tese anti-migration features to prevent the dislocation or unwanted movement of the volume restoration element 10.

The volume restoration element 10 of the present invention advantageously relieves e pre-operative impingement (compression) of the spinal nerves 18, which results in pain relief for the patient and/or improved motor and/or sensory nerve functions (if previously impaired due to nerve root compression). The volume restoration element 10 also advantageously restores the facet joint to a normal or near-normal physiologic state, by loading the facet joint and/or returning the superior and inferior articulating facets 36, 28 into proper position. This is helpful in that it reverses or minimizes any facet imbrication that may have existed in the pre-operative stenotic condition. The volume restoration element 10 of the present invention also advantageously causes distraction osteogenesis over time, which means the boney aspects constituting the periphery of the IVF 20 which are in contact with the volume restoration element 10 may gradually migrate or move as a result of the force being exerted by the volume restoration element 10. In so doing, the volume of the IVF 20 may actually increase over time, further ensuring protection from nerve compression, facet imbrication, and undesirable facet loading.

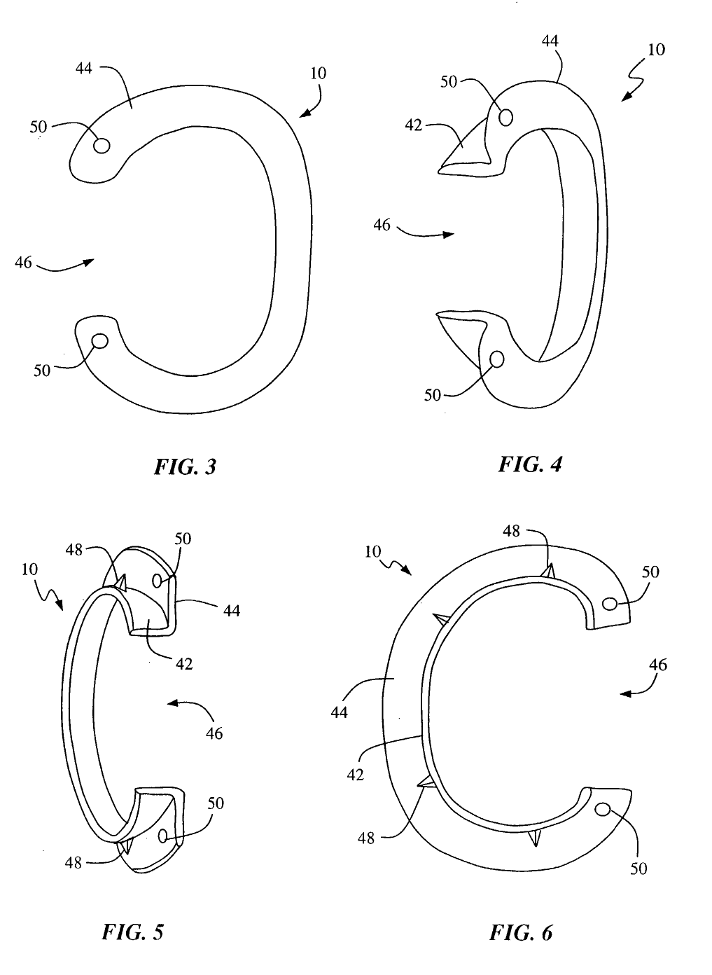

FIGS. 3-6 illustrate in greater detail the volume restoration element 10 according to the embodiment shown in FIGS. 1-2. The volume restoration element 10 includes a generally curved expansion member 42 and a flange 44 extending generally perpendicularly from the expansion member 42. Expansion member 42 includes an open region 46 and at least one anti-migration feature 48. Anti-migration features 48 may include any number of projections or mechanisms designed to prevent the volume restoration element 10 from rotating or dislocating relative to the IVF 20 once implanted, including but not limited to spurs, spikes, grooves, and/or ridges. Flange 44 may extend along the entire length of expansion member 42, and serves to prevent the lateral migration of volume restoration element 10 into the spinal canal. It is contemplated that flange 44 may be split into several regions such that flange 44 is not one continuous member but rather several separated by predetermined distances. Flange 44 may also contain one or more apertures 50. The apertures 50 are configured to be coupled to an insertion device 52, as will be discussed in greater detail below. Apertures 50 may also be configured to receive at least one bone engaging screw (not shown) to secure the volume restoration element 10 in position within the IVF 20. Although shown as located on flange 44, apertures 50 may also be oriented at any point along the expansion member 42 if desired. Also, although flange 44 is depicted as protruding in a generally perpendicular manner from the expansion member 42, the flange 44 may be oriented in any manner suitable for facilitating engagement with the bone, including but not limited to (and by way of example only) a generally curved orientation.

The volume restoration element 10 of the present invention may be provided in any number of suitable configurations in addition to that shown and described with reference to FIGS. 1-6 without departing from the scope of the present invention. Moreover, it is within the scope of the present invention to combine the features and aspects of any one embodiment with one or more features and aspects of any other embodiment or embodiments. With this general appreciation in hand, various exemplary embodiments of the present invention will now be described.

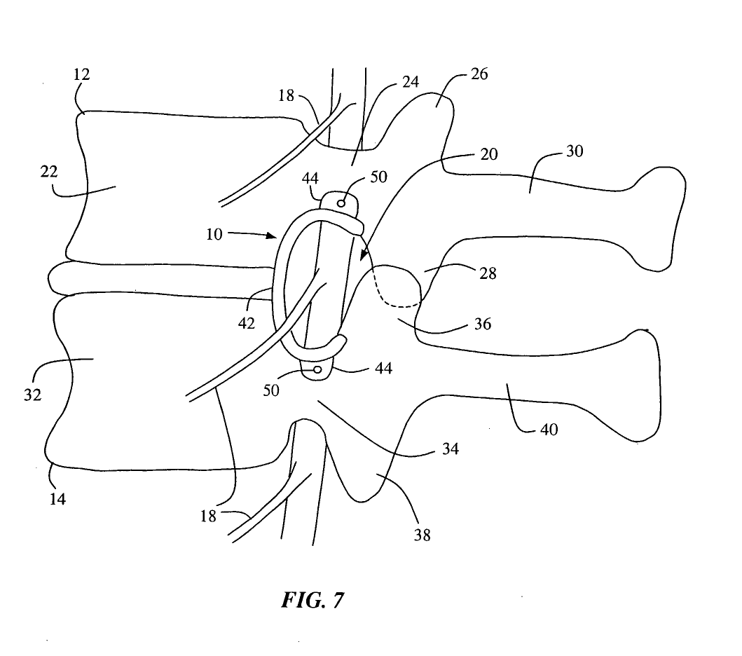

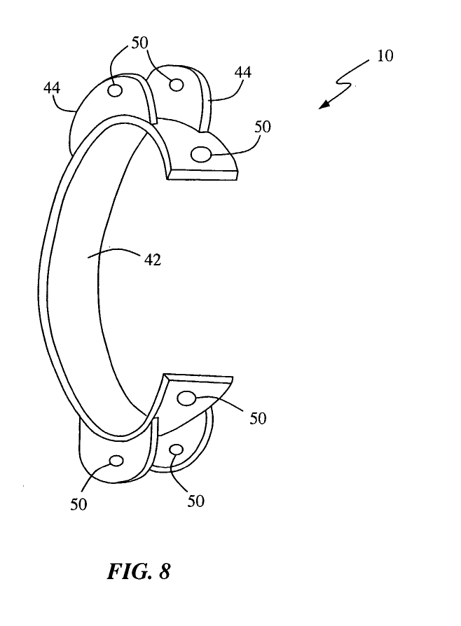

FIGS. 7-8 illustrate a volume restoration element 10 according to another embodiment of the present invention. In this instance, as best viewed in FIG. 8, the expansion member 42 is provided with at least one pair of flanges 44 on either side of the expansion member 42. By way of example only, a first pair of flanges 44 is provided along the superior aspect of the volume restoration element 10 and a second pair of flanges 44 is provided along the inferior aspect of the volume restoration element 10. Apertures 50 may be provided in one or more of each pair of flanges 44 for the purpose of receiving fixation screws (not shown) to secure the volume restoration element 10 in position after implantation, as well as for coupling to an insertion device (not shown) to assist in introducing the volume restoration element 10 into the IVF 20.

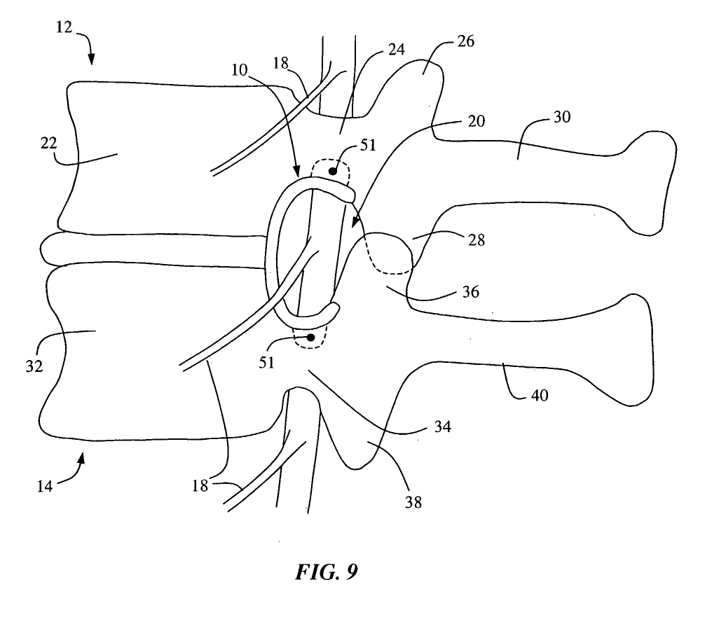

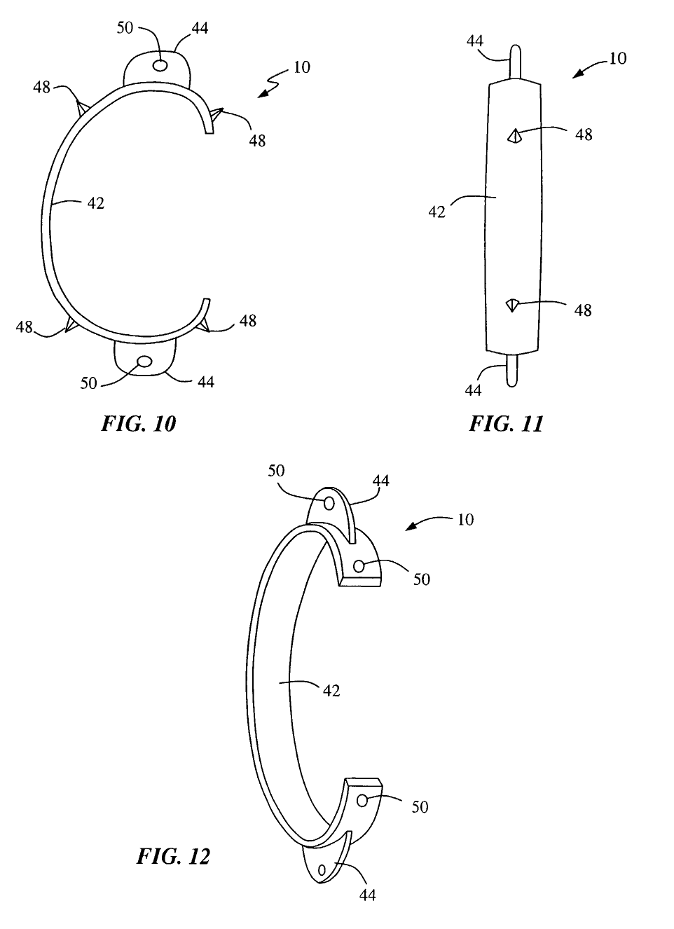

FIGS. 9-12 illustrate a volume restoration element 10 according to a still further embodiment of the present invention. In this embodiment, the expansion member 42 is equipped with at least one flange 44, where each such flange 44 is positioned a predetermined distance medially with respect to the edge of the expansion member 42. By way of example only, as best viewed in FIG. 11, each flange 44 is positioned in the approximate middle of width of the expansion member 42. In use, as best viewed in FIG. 9, the superior flange 44 is disposed within a groove or recess (shown in phantom) formed within the superior pedicle 24 and the inferior flange 44 is disposed with a groove or recess (shown in phantom) formed within the inferior pedicle 34. Apertures 50 may be provided in one or more of each pair of flanges 44 for the purpose of receiving a fixation element 51 shown in FIG. 9) to secure the volume restoration element 10 in position after plantation, as well as for coupling to an insertion device (not shown) to assist in producing the volume restoration element 10 into the IVF 20. Each fixation element 51 y comprise any number of suitable articles for securing the flanges 44 within the esses, including but not limited to a pin, a dowel, a plug, and a bone screw. It will be appreciated that the flanges 44 may be located at additional (or alternative) locations und the expansion member 42 to correspond with grooves formed in other locations ng the periphery of the IVF 20 (e.g. vertebral bodies 22, 32, disc 16, facets 28, 36).

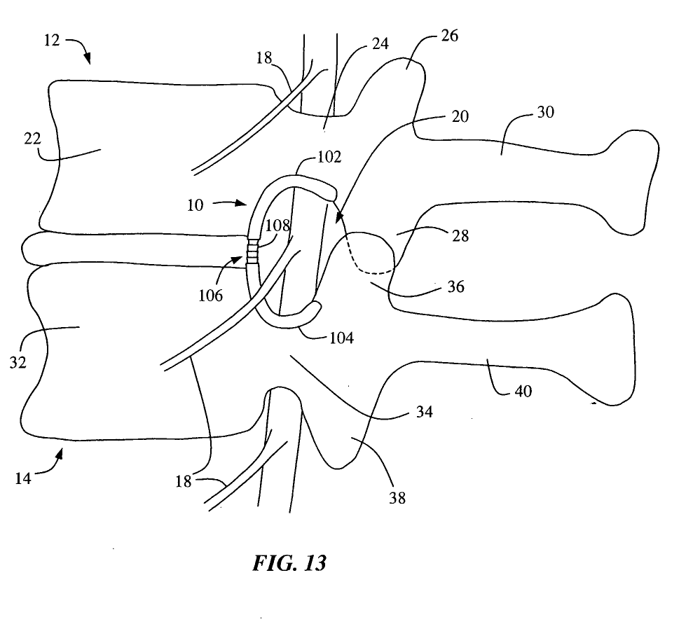

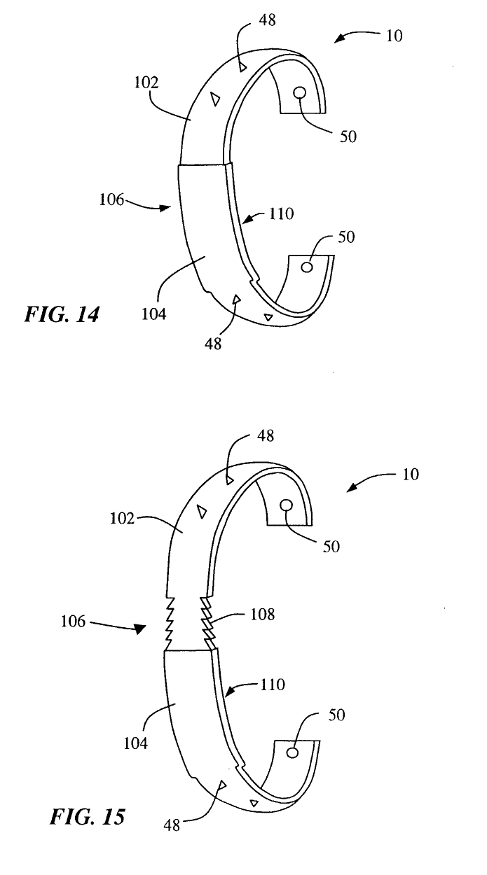

FIGS. 13-14 illustrate a yet another embodiment of the present invention, comprising an expandable volume restoration element 10. Volume restoration element 10 generally curved as with the previous embodiment but is constructed from a first expansion member 102 slidably coupled to a second expansion member 104. First expansion member 102 may include an extension region 106 extending at least partially ng the perimeter of first expansion member 102. Preferably, extension region 106 includes a locking feature configured to maintain the volume restoration element 10 in an ended position once the user has extended the volume restoration element 10 to a ired position. By way of example only, the locking feature may consist of a plurality of ches or ridges 108 configured to provide a ratchet-like interaction with the second expansion member 104. Second expansion member 104 includes a hollow region 110 dimensioned to slidably receive at least a portion of first expansion member 102. Second expansion member 104 further includes a complimentary locking feature (not shown) dimensioned to cooperate with the locking feature of the extension region 106. Each of the first and second expansion members 102, 104 may include at least one aperture 50 for management with an insertion device and/or for receiving a bone screw. Optionally, expansion members 102, 104 may be provided with any number, type and/or combination anti-migration features 48 and flanges 44 as discussed above in relation to volume oration element 10.

In use, as shown in FIG. 13, the volume restoration element 10 may be inserted into IVF 20 via any suitable means, including but not limited to those described in greater detail below. During insertion, the extension region 106 of the first expansion member 102 preferably oriented substantially within the hollow region 110 of the second expansion number 104, as shown in FIG. 13. Once volume restoration element 10 is position in a desirable location, first expansion member 102 is slidably advanced such that extension ion 106 emerges at least partially from the hollow region 104 and the volume oration element 10 fully engages the perimeter of the IVF 20. To accomplish this, the r may employ any expansion device (not shown) capable of causing the first expansion number 102 to extend from the second expansion member 104. By way of example only, extender device may include a sequential dilation system (e.g. cannulae), a mechanical nder having a handle portion, an elongated shaft and a stent engagement portion, and/or a balloon dilator. By way of further example, a balloon dilator may be provided ing a generally rigid portion configured to allow expansion in a limited number (e.g.)) of directions to further control the expansion of the volume restoration element 10 to prevent encroachment on any nearby nerve roots 18. Volume restoration element 10 y be configured such that the amount of extension required in a given vertebral level is determined by the size of the IVF 20. In the alternative, volume restoration element 10 y be provided in a fully adjustable configuration such that the volume restoration element 10 may be appropriately sized at the time of insertion and/or extension as required the particular size and shape of the IVF 20.

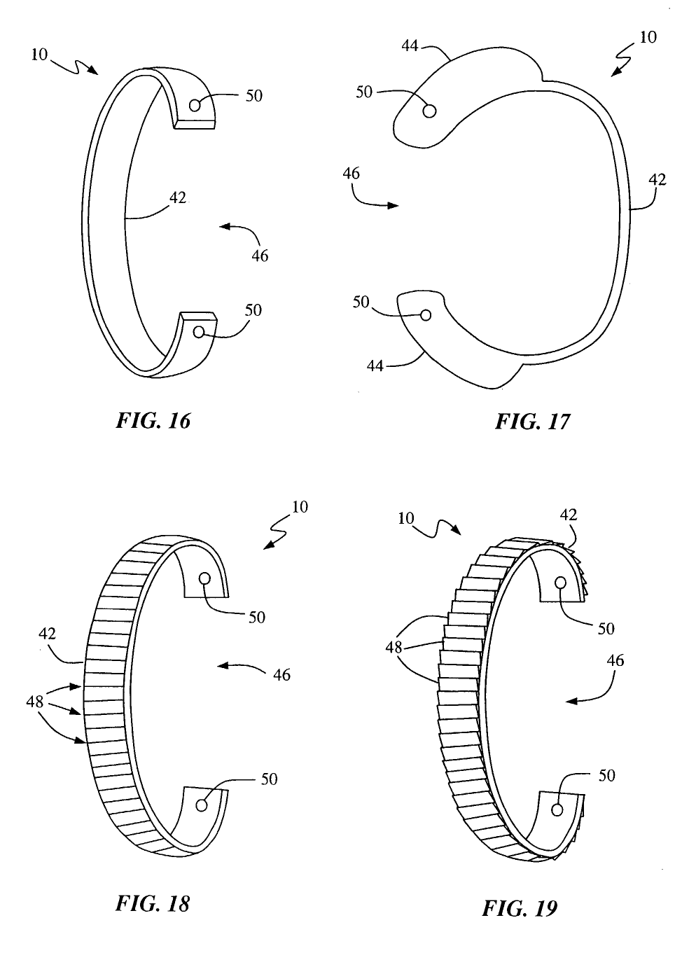

FIGS. 16-21 illustrate various additional embodiments of the volume restoration element 10 of the present invention, all of which are within the scope of the present invention individually and in combination (in whole or in part) with any other embodiment embodiment described herein.

FIG. 16 illustrates a volume restoration element 10, wherein the expansion member is without flanges but equipped with a pair of apertures 50 located near each end of the expansion member 42. Although shown with two apertures 50 (for insertion tool engagement and/or fixation screw introduction), it will be appreciated that any number of additional apertures 50 may be provided at any point along expansion member 42 for either purpose. It will also be appreciated that the expansion member 42 may be provided without apertures 50 so long as an additional fixation mechanism is provided for securing the expansion member 42 within the IVF 20, such as (by way of example only) the use of adhesive or bonding material placed between the volume restoration element 10 and the bone constituting the periphery of the IVF 20 and/or the use of suture or cabling extending around the volume restoration element 10 and through or around the bone or other structures constituting and/or surrounding the IVF 20.

FIG. 17 illustrates the volume restoration element 10 having at least two flanges 44, which are (by way of example only) located near each end of expansion member 42. Although shown with two flanges 44, it will be appreciated that any number of additional flanges 44 may be provided at any point along the expansion member 42. Also, although shown with two apertures 50 (for insertion tool engagement and/or fixation screw introduction), it will be appreciated that each flange 44 may be provided with one or more additional apertures 50 for either purpose.

FIGS. 18-19 illustrate the volume restoration element 10 with two alternate manners of providing anti-migration features 48. In FIG. 18, the anti-migration features 48 comprise are a series of grooves that create a friction between the volume restoration element 10 and the bone constituting the periphery of the IVF 20. In FIG. 19, the anti-migration features 48 comprise a series of ridges that create a friction between the volume restoration element 10 and the bone of the IVF 20, but also increase the anti-migration properties by slightly digging into the bone of the IVF 20.

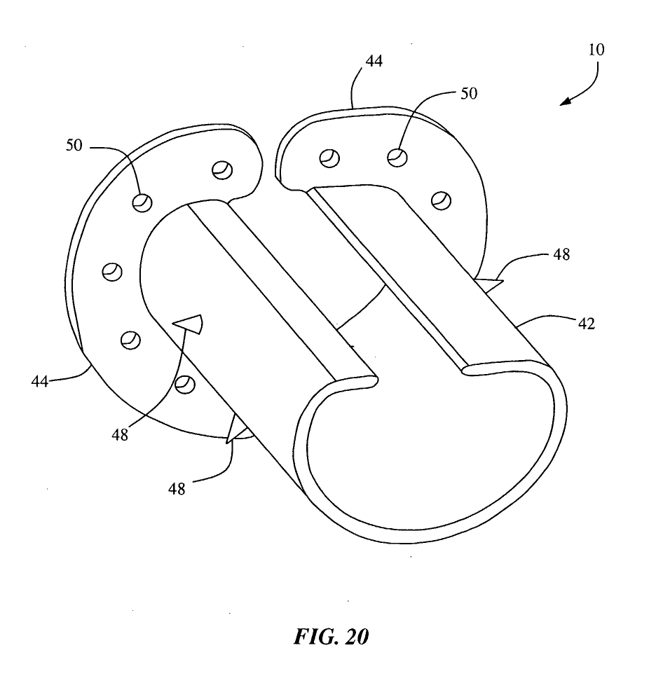

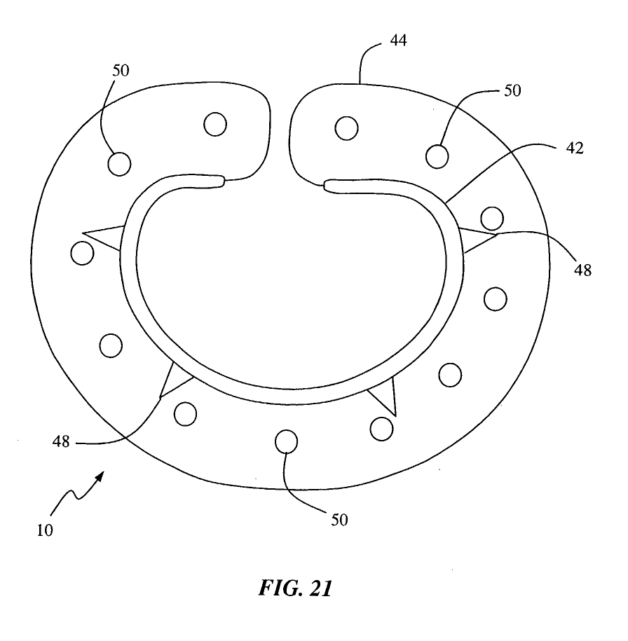

FIGS. 20-21 illustrate an alternate embodiment wherein the volume restoration element 10 is provided having a greater width than in the “narrow” embodiments shown and described with regards to FIGS. 1-19 above, which are dimensioned to extend only partially into the IVF 20. However, in some instances it may be desirable to dimension the volume restoration element 10 where the expansion member 42 has a greater width ension, such as shown in FIGS. 20-21. The expansion member 42 may be equipped h additional anti-migration features 48 and/or additional apertures 50 distributed within flange 44. The increased width of this embodiment allows the expansion member 42 to gress further into the IVF 20, which may be desirable depending on the size of the ient, the expansion forces required to restore and maintain the volume of the fVF 20, other factors. Although not shown, the volume restoration element 10 of FIGS. 20-21 y provided with one or more additional flange (not shown) opposite to flange 44 to w for the anchoring of the volume restoration element 10 at one or more points along medial end of the IVF 20, akin to the embodiment shown in FIGS. 7-8.

The volume restoration elements of the present invention may be introduced into IVF 20 in any number of suitable manners, including but not limited to the use of the rtion instruments and preparation instruments described below.

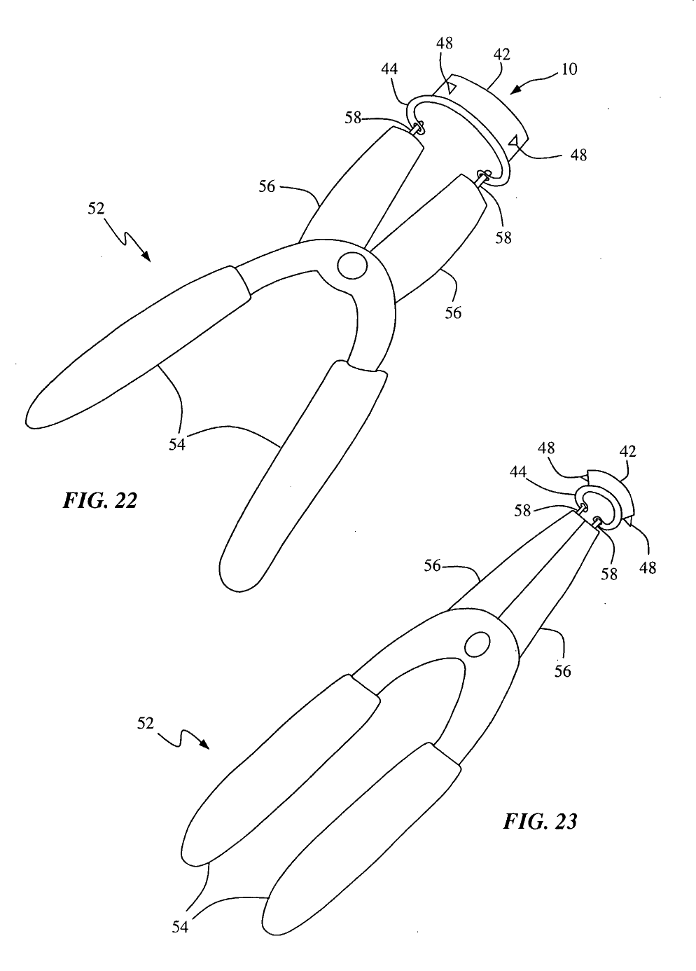

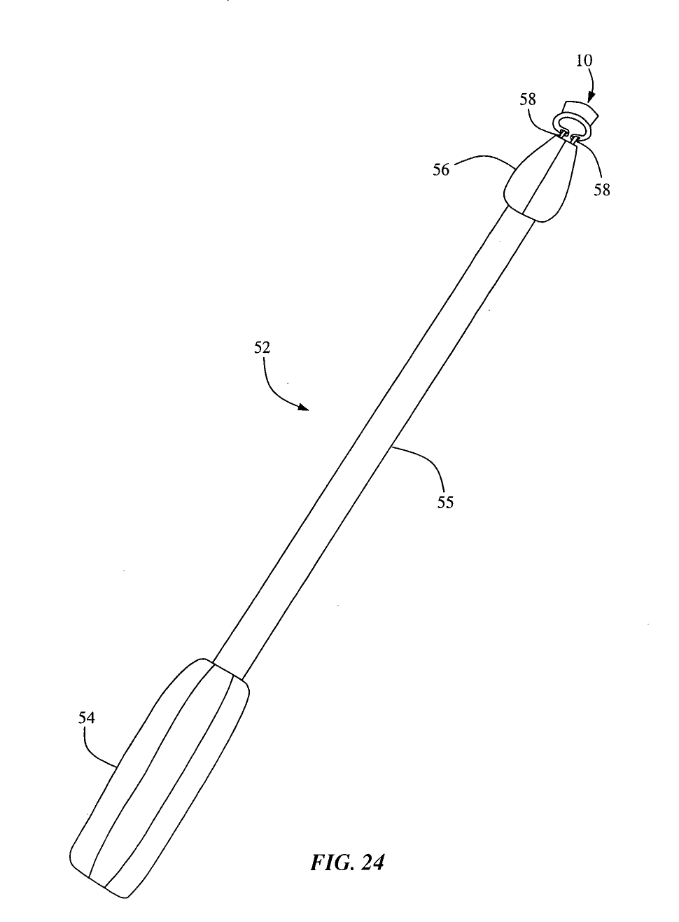

FIGS. 22-24 illustrate a two-armed insertion device 52 for inserting and deploying of the volume restoration elements 10 described herein which are equipped with at t two apertures 50 according to the present invention. Insertion device 52 includes a handle feature 54, a pair of insertion arms 56, and a pair of aperture-engaging features 58. the embodiment of FIG. 24, insertion device 52 includes an elongated shaft 55 extending between the handle feature 54 and the insertion arms 56 to facilitate insertion though a ill surgical corridor. Elongated shaft 55 and/or insertion arms 56 may be straight, led, or curved as desired to facilitate insertion of volume restoration element 10 into the 20 by any known surgical approach. In either embodiment, aperture-engaging features engage the volume restoration element 10 by extending through the apertures 50 on the flange 44. Although not shown, it will be appreciated that the aperture-engaging features may also be used to engage apertures 50 formed on the expansion member 42. In use, volume restoration element 10 is fitted onto insertion device 52 through the association described above. To facilitate this association, arms 56 are spread apart to accommodate matching of aperture-engaging features 58 with apertures 50. Once the volume restoration element 10 is secured onto the insertion device 52, the handle feature 54 is vated, causing arms 56 to come together and forcing volume restoration element 10 into ntracted state, as shown in FIG. 23 or 24. Volume restoration element 10 is then rted through a proper operative corridor into position within the IVF 20. In one embodiment, the volume restoration element 10 may be inserted by a direct approach ugh an operative corridor. To accomplish this insertion, spinal nerve 18 may have to etracted using common nerve retraction procedures. Once positioned as desired, the dle feature 54 is released, causing arms 56 to spread apart and allowing the volume oration element 10 to spring back to assume its original state.

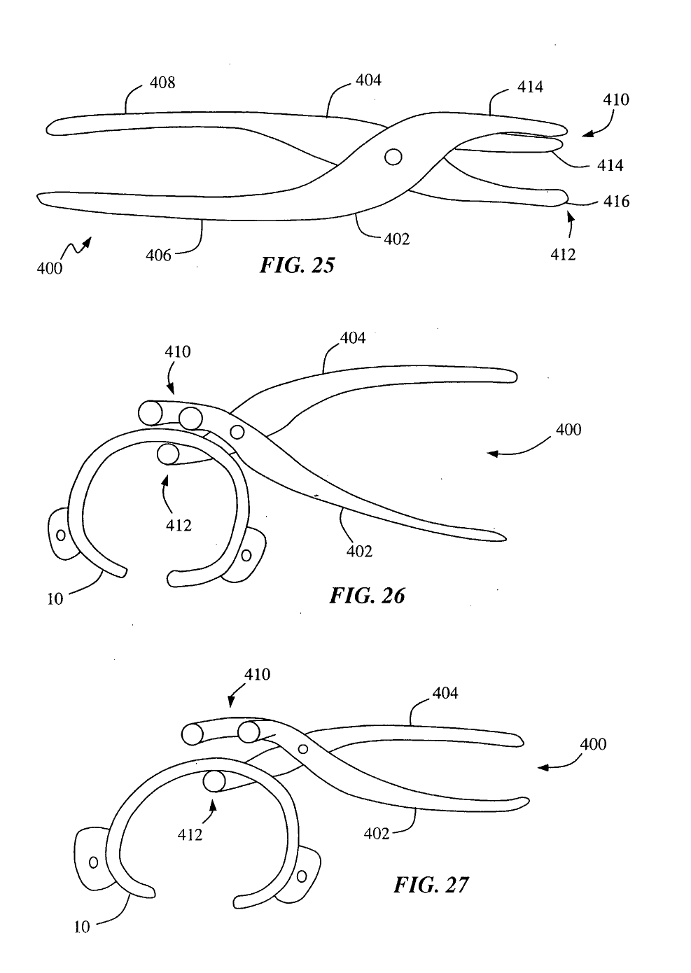

FIGS. 25-27 illustrate a three-arm insertion device 400 for inserting and deploying of the volume restoration elements 10 described herein, regardless of whether they apertures 50. Insertion device 400 generally consists of a three-pronged pliers commonly used in dental applications. Insertion device 400 has first and second generally red elongated members 402, 404 each having a handle region 406, 408 and an agement region 410, 412. Engagement region 410 may have a pair of prongs 414 figured to engage a volume restoration element 10 of the present invention on one side expansion member 42. Engagement region 412 may have a single prong 416 configured gage volume restoration element 10 on an opposite side of expansion member 42. ing insertion, volume restoration element 10 is coupled to insertion device 400 as shown in FIG. 26. Insertion device 400 maintains volume restoration element 10 in a ively closed configuration to facilitate insertion into the IVF 20. Once volume restoration element 10 is positioned as desired, the user releases the volume restoration element 10 by opening the handle regions 406, 408, which causes engagement regions 410, to move away from each other. The volume restoration element 10 is then capable of inding into its open and expanded position within IVF 20. The volume restoration element 10 may then be secured within the IVF 20 by any suitable means, including but not ted to bone screws pins, dowels, and the like.

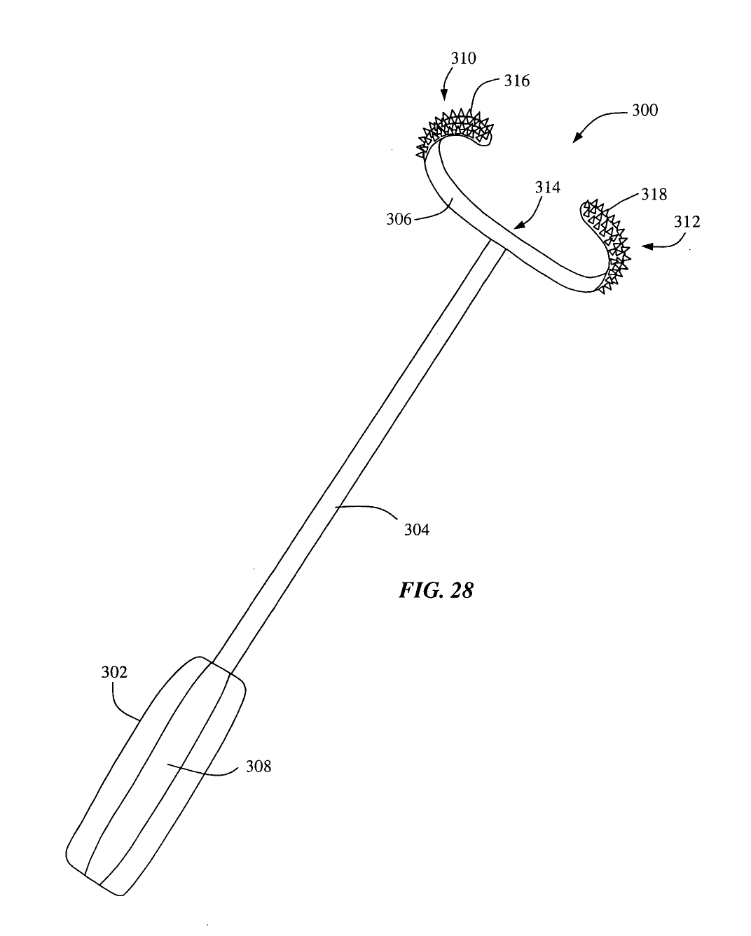

FIG. 28 illustrates an exemplary recess creation device 300 for fabricating grooves within bone dimensioned to receive flanges 208 of volume restoration element as shown in FIGS. 9-12 according to the present invention. The recess creation device 300 may include a handle 302, an elongated shaft 304, and a cutting member 306 located at the distal of the elongated shaft 304. Handle 302 may have any shape or features desirable for a comfortable and functional interaction with a user. By way of example only, handle 302 may be generally cylindrical and have a plurality of gripping features 308 to provide friction between the handle 302 and the user's hand. Preferably, elongated shaft 304 has a generally circular cross-section, but it is contemplated that the elongated shaft 304 may have a cross-section consisting of any geometric shape, including but not limited to generally oval and/or generally polygonal. Cutting member 306 may have any shape capable of facilitating the cutting of bone to create a recess to receive each flange 44 of the volume restoration element 10 as shown in FIGS. 9-12. By way of example only, cutting member 306 may be fashioned as a generally crescent or “C” shaped member having first and second cutting regions 310, 312 separated by an attachment region 314. First and second cutting regions 310, 312 may each include a plurality of teeth 316, 318 or other suitable cutting feature distributed along the exterior of the region. Cutting member 306 may be attached to the elongated shaft 304 at the attachment region 314 by any means desirable, including but not limited to (and by way of example only) molding, welding, screws, pins, dowels, threaded engagements, or snap-fit engagements. First and second cutting regions 310, 312 may also be hingedly attached to the elongated shaft 304 at attachment region 306 and configured to be movable from a first contracted position (e.g. during insertion through an operative corridor) to a second fully extended position (e.g. shown in FIG. 28) prior to preparing the bone.

To use the recess creation device 300, a user must first establish an operative corridor to the surgical target site. Once this is accomplished, the user may then insert the recess creation device 300 into the corridor such that the cutting member 306 is advanced within the IVF 20 with the cutting regions 310, 312 are positioned adjacent to the target bone segment. By way of example only, the target bone segment may consist of pedicle 24, 34 of first and second vertebrae 12, 14 as shown in FIG. 9. At this point the user may rotate the handle 302 back and forth such that the cutting member 306 is caused to rotate and the teeth 316, 318 engage and dig into the pedicle 310, 312. Once a groove of appropriate depth is created in this manner, the recess creation device 300 may be removed from the operative corridor. At all times, care should be taken to avoid contact with the nearby nerve root 18. Alternatively, nerve root 18 may be retracted prior to insertion of the saw 300.

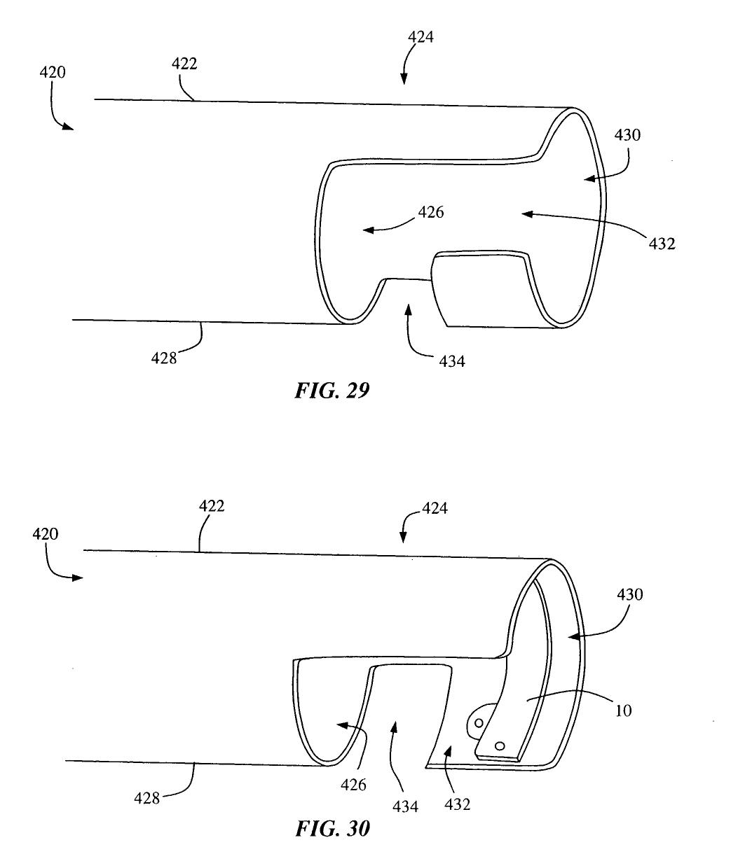

FIGS. 29-34 illustrate an inserter 420 for inserting and deploying any of the volume restoration elements 10 described herein (preferably without flanges 44 extending perpendicularly from the expansion member 42) according to the present invention. The inserter 420 is dimensioned to insert volume restoration element 10 without retracting the nerve root 18. Inserter 420 may include a generally tubular member 422 having a distal region 424. Generally tubular member 422 has a lumen 426 extending at least partially the length of tubular member 422, and a housing member 428 surrounding the lumen 426. Generally tubular member 422 has a distal opening 430 and first and second cutout regions 432, 434 located at distal region 424. First cutout region 432 is adjacent to the distal opening 430 and extends in a proximal direction along the tubular member 422. Second cutout region 434 is immediately adjacent and generally perpendicular to the first cutout region 432 and extends at least partially around the circumference of the tubular member 422. When viewed together, first and second cutout regions 432, 434 may generally form an “L” shaped cutout region as shown in FIG. 29. First and second cutout regions 432, 434 may be of varying widths and lengths depending on the particular needs of the user, and are both dimensioned to receive a portion of a nearby nerve root 18. The housing member 428 may have generally rounded edges where it forms the perimeter of first and second cutout regions 432, 434 so as to not damage the nerve root 18 during use.

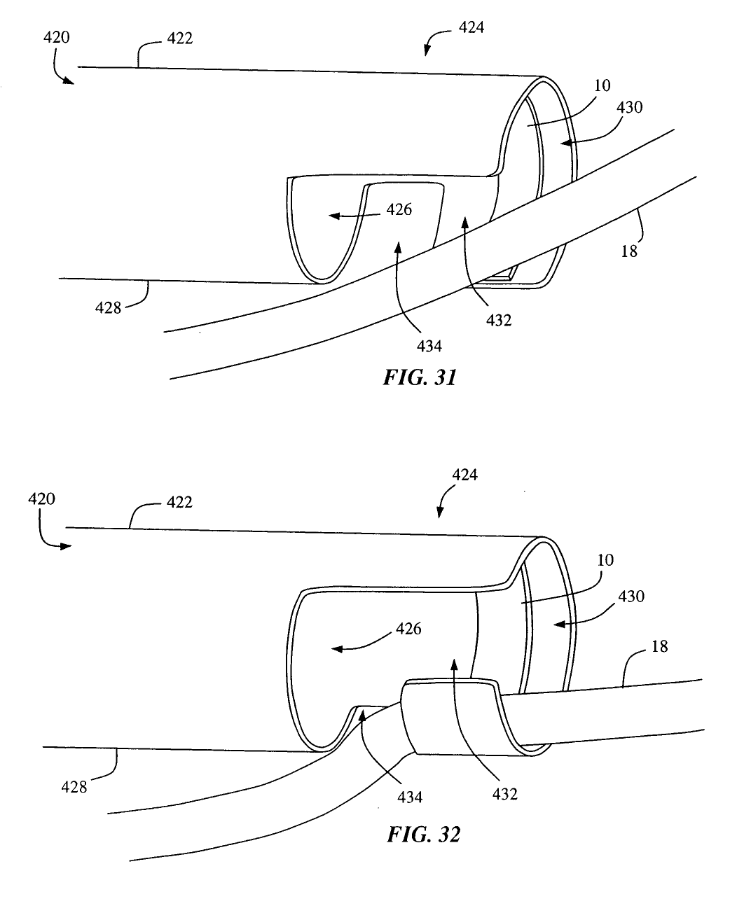

In use, a volume restoration element 10 is first loaded into lumen 426 at the distal region 424 of the inserter 420 as shown by way of example only in FIG. 30. As shown, the inserter 420 is configured to approach the IVF 20 from the left. Although not shown, it is to be understood that inserter 420 may be configured to approach the IVF 20 from the right side. In such a case the cutout regions 432, 434 would be configured in a mirror image orientation to that shown in FIGS. 31-36. Furthermore, inserter 420 may be used with any embodiment of the volume restoration element 10 described above without departing from the scope of the present invention. After volume restoration element 10 has been loaded into the inserter 420, the distal region 424 is advanced along an established operative corridor toward the target IVF 20 (for example shown in FIG. 1).

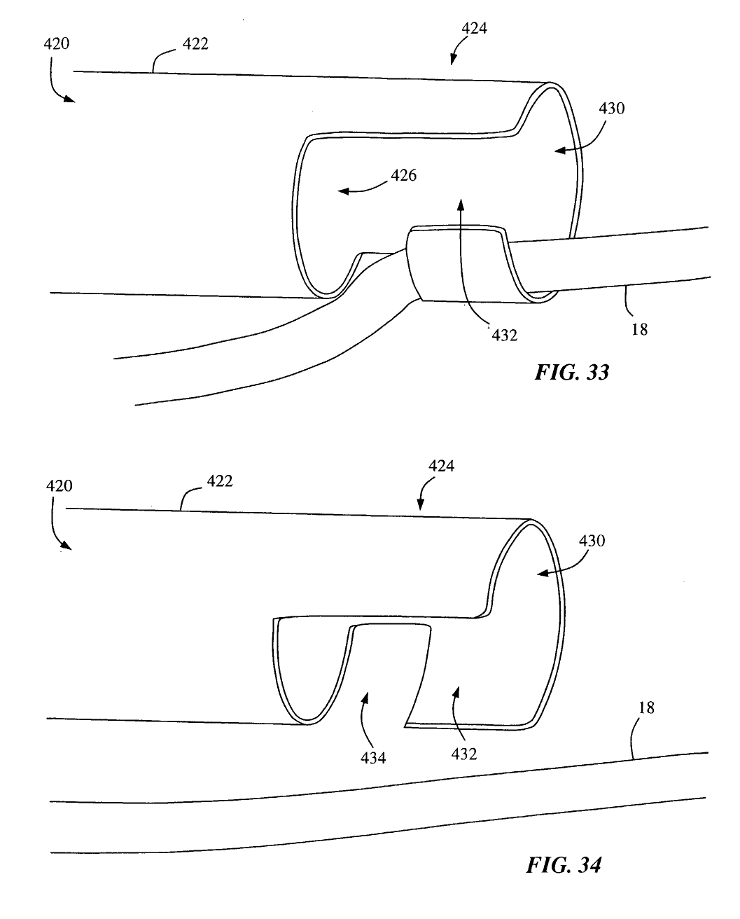

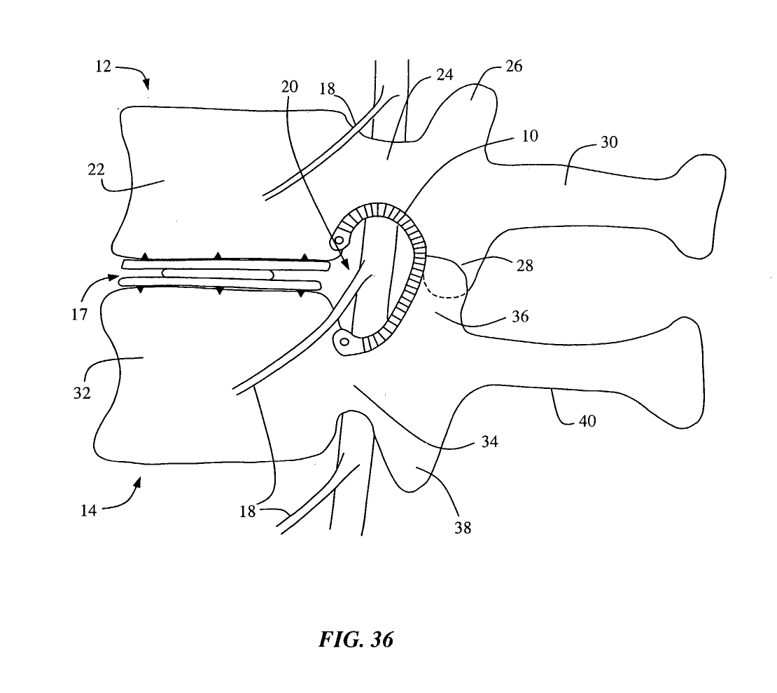

Prior to advancing into IVF 20, the distal region 424 may encounter the nerve root 18 protruding from the IVF 20. To continue inserting the volume restoration element 10 without retracting the nerve root 18, the distal region 424 may be rotated such that the nerve root 18 enters the lumen 426 via the first cutout region 432 and the distal opening 430, as shown in FIG. 33. The user may then rotate the inserter 420 such that the nerve root 18 interacts with the second cutout region 434, as shown in FIG. 34. At this point the nerve root 18 is inside the lumen 426 at the distal region 424, and also inside the perimeter of the volume restoration element 10. As such, the volume restoration element 10 is positioned for insertion into the IVF 20. To accomplish this, an ejection device (not shown) is employed to eject the volume restoration element 10 through distal opening 430 of inserter 420 and into the desired location in the IVF 20. The ejection device may consist of an ejection member traversing lumen 426 to force out the volume restoration element 10 and may be an integral part of inserter 420 or be provided on a stand-alone instrument. Once the volume restoration element 10 has been ejected, the inserter 420 may be rotated in an opposite direction to disengage the nerve root 18 from the lumen 426, as shown in FIG. 36. As the inserter 420 is rotated, the nerve root 18 will first exit second cutout region 434, followed by the first cutout region 432 and the distal opening 430. Once the nerve root 18 has been disengaged, the inserter 420 may be removed from the operative corridor.

The volume restoration element 10 of the present invention is extremely versatile, in that it may be used as a stand-alone device as shown and described above, as well as in conjunction with other spinal implants. As a stand-alone device, the volume restoration element 10 of the present invention will be the only type of device solution implanted in the patient, and may be employed in only one of IVF 20 of the pair for each vertebral level (so-called “unilateral volume restoration” such as may be desirable, by way of example only, for scoliosis correction) and/or in each IVF for a particular vertebral level (so-called “bi-lateral volume restoration”). The volume restoration element may be employed at a single vertebral level or in multiple vertebral levels (adjacent or non-adjacent).

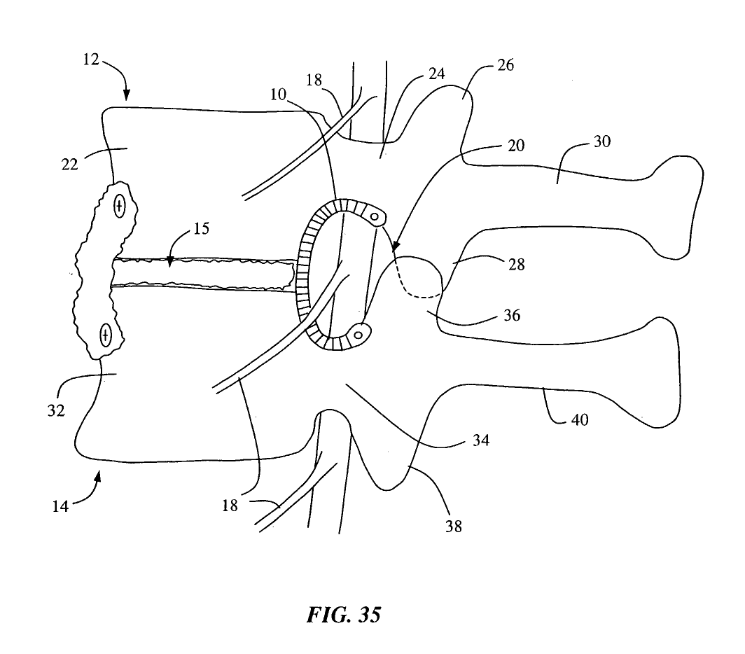

When used in conjunction with other spinal implants, the volume restoration element of the present invention may advantageously supplement or augment the function of the other devices. FIG. 35 illustrates, by way of example only, a volume restoration element 10 in use with an exemplary nucleus replacement implant 15 implanted in between the vertebral bodies 22, 32 in accordance with the present invention. FIG. 36 illustrates, by way of example only, a volume restoration element 10 in use with an exemplary total disc replacement implant 17 implanted in between the vertebral bodies 22, 32 in accordance with the present invention. It will be appreciated that, although shown in use with specific types of nucleus replacement and total disc replacement implants, the volume restoration element 10 of the present invention may be employed with any additional types of nucleus replacement and total disc replacement implants, as well as any additional spinal implants (such as facet replacement systems, dynamic stabilization systems, spinous process spacing systems, and vertebral body repair devices or systems such as kyphoplasty), without departing from the scope of the present invention.

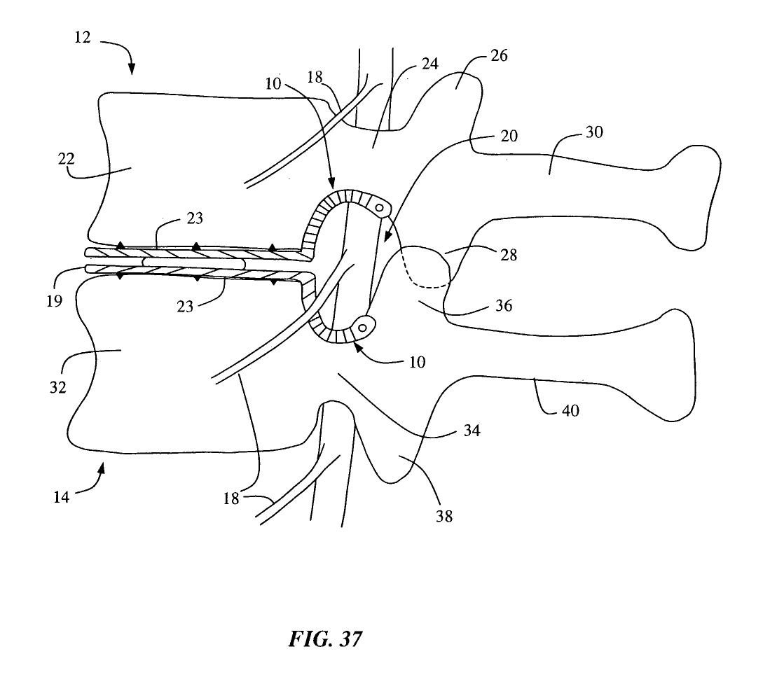

It is also within the scope of the present invention to provide or incorporate a volume restoration element 10 of the present invention (in whole or in part) into any of a variety of other spinal implants, so as to benefit from the advantages of the volume restoration element of the present invention in addition to those of the other spinal implants. FIG. 37 illustrates, by way of example only, an exemplary total disc replacement implant 19 wherein each endplate 23 has a volume restoration element 10 extending from a posterior aspect thereof. According to the present invention, the total disc replacement implant 19 may be provided with one or more of the volume restoration elements 10 without departing from the scope of the present invention, with each volume restoration element 10 serving to directly restore the volume of the IVF 20 according to the present invention. It will be appreciated that, although shown in use with specific type of total disc replacement implant, the volume restoration element 10 of the present invention may be employed with any additional types of total disc replacement implants, as well as any additional spinal implants (such as nucleus replacement implants, facet replacement systems, dynamic stabilization systems, and spinous process spacing systems), without departing from the scope of the present invention.

The volume restoration element of the present invention may also be used at vertebral levels above or below such other spinal implants, including fusion implants, to reduce the incidence of so-called adjacent level disease and/or add to the overall structural stability of the spine.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the invention is to cover all modifications, equivalents, and alternatives falling within the spirit and scope of the invention as defined herein.

1. An apparatus for restoring volume of a stenotic intervertebral foramen of a spine, comprising:
at least one volume restoration element dimensioned to be received within and adapted to restore the volume of an intervertebral foramen in a spine.

2. The apparatus of claim 1, wherein said volume restoration element has at least one anti-migration feature.

3. The apparatus of claim 1, wherein said volume restoration element has at least one flange.

4. The apparatus of claim 1, wherein said volume restoration element has at least one aperture formed therein.

5. The apparatus of claim 3, wherein said at least one flange is disposed on an expansion member.

6. The apparatus of claim 5, wherein said at least one flange is disposed on at least one of an edge of said expansion member and an interior location on said expansion member.

7. The apparatus of claim 1, wherein said volume restoration element is dimensioned to be received within an intervertebral foramen in at least one of the cervical spine, thoracic spine, and lumbar spine.

8. The apparatus of claim 1, wherein said volume restoration element may be applied at least one of unilaterally and bilaterally.

9. The apparatus of claim 1, wherein said volume restoration element may be incorporated into the manufacture of another type of spinal implant.

10. The apparatus of claim 9, wherein said other spinal implant comprises at least one of a total disc replacement, nucleus replacement, and facet replacement.

11. The apparatus of claim 1, wherein said volume restoration element including at least one expansion member adapted to expand from a first position to a second position.

12. A method for restoring volume of a stenotic intervertebral foramen of a spine, comprising:
introducing a volume restoration element within said intervertebral foramen to restore the volume of said foramen.

13. The method of claim 12, wherein said volume restoration element includes at least one anti-migration feature.

14. The method of claim 12, wherein said volume restoration element includes at least one flange.

15. The method of claim 14, wherein said at least one flange is provided on an expansion member.

16. The method of claim 15, wherein said at least one flange is provided on at least one of an edge and an interior portion of said expansion member.

17. The method of claim 12, wherein said volume restoration element includes at least one aperture formed therein.

18. The method of claim 12, wherein said volume restoration element is dimensioned to be received within an intervertebral foramen in at least one of the cervical spine, thoracic spine, and lumbar spine.

19. The method of claim 12, wherein said volume restoration element may be applied at least one of unilaterally and bilaterally.

20. The method of claim 12, wherein said volume restoration element may be incorporated into the manufacture of another type of spinal implant.

21. The method of claim 20, wherein said other spinal implant comprises at least one of a total disc replacement, nucleus replacement, and facet replacement.

22. The method of claim 12, further comprising:
distracting said intervertebral foramen from a compressed state to a restored state.

23. The method of claim 22, wherein the step of distracting said intervertebral foramen comprises using a distraction instrument to distract said intervertebral foramen.

24. The method of claim 22, wherein the step of distracting said intervertebral foramen comprises expanding said volume restoration element from a first position to a second position.
